(19) United States
(12) Patent Application Publication
(10) Pub. No.: US 2026/0185967 A1
(43) Pub. Date: Jul. 2, 2026

(54) METHOD FOR DETECTING NATURAL FLAVONOID SELENIUM

(71) Applicant: Shanghai Aiqi Pharmaceutical Co., Ltd., Shanghai (CN)

(72) Inventors: Kunyuan Song, Shanghai (CN); Weiwei Chen, Shanghai (CN)

(21) Appl. No.: 19/001,569

(22) Filed: Dec. 26, 2024

(51) Int. Cl. G01N 30/14; G01N 30/02; G01N 30/16

(52) U.S. Cl. CPC G01N 30/14; G01N 30/16; G01N 2030/027

(57) ABSTRACT

A method for detecting natural flavonoid selenium, including the following steps: S1: preparing mobile phase; S2: preparing test solution; S3: testing the test solution, the present disclosure is applicable to the field of pharmaceutical technology and provides the detection method for natural flavonoid selenium to ensure direct quantification of a quality control in drug development and application. Through a standard curve, a compensation effect of a dual ternary pump can be utilized to render a response value of each substance passing through a detector correspond to an amount of substance, without being affected by the fluidity gradient and without the need to prepare standard samples again.

### TECHNICAL FIELD

The present disclosure relates to the field of pharmaceutical technologies, and in particular, to a method for detecting natural flavonoid selenium.

### BACKGROUND

Flavonoids are widely distributed in various plants and have effects such as anti-tumor, antiviral, antioxidant, free radical scavenging, enhancing immunity, protecting the cardiovascular system, lowering blood sugar, delaying aging, antibacterial, anti-inflammatory, regulating capillary fragility and permeability. In recent years, many research results have shown that flavonoids mostly exert their effects through synergy with essential elements for life.

Selenium is an essential trace element for the human body, with various biological activities such as antioxidants, anticancer, anti-cancer, bone marrow hematopoietic protection, anti-aging, etc. It also has detoxifying effects on some heavy metal elements (such as mercury, arsenic, silver, etc.).

In recent years, synthesized organic selenium compounds, such as selenized chitosan, selenoproteins, selenium polysaccharides, selenized carrageenan, selenized tea polyphenols, and other small or biomacromolecule selenium compounds, have significantly higher biological activities in anti-tumor, cardiovascular disease treatment, anti-aging, and immune enhancement compared to corresponding compounds without selenization. Natural flavonoid selenium is a selenized flavonoid compound that utilizes unique process compounds and has unique chemical and biological properties, while solving the technical barriers of bioavailability and toxicity.

At present, there is relatively little research on flavonoid selenium. In 2010, Zhou Wenfu et al. published “Solid liquid phase transfer synthesis and structural analysis of medicinal compounds of flavonoid selenium from Caoshanhu”, which mainly introduced the synthesis method and structural analysis of flavonoid selenium from Caoshanhu. However, there was no relevant research on the liquid-phase analysis and quantitative analysis of the flavonoid selenium complexes from Caoshanhu.

### SUMMARY

The present disclosure provide a method for detecting natural flavonoid selenium to overcome the shortcomings of the prior art.

To achieve the above objectives, the present disclosure adopts the following technical solution.

The method for detecting natural flavonoid selenium, including the following steps:
S1: preparing mobile phase;
S2: preparing test solution;
S3: testing the test solution.

In some embodiments of the present disclosure, in step S1, the mobile phase includes a mobile phase A, a mobile phase B, and a 50% acetonitrile solution.

In some embodiments of the present disclosure, in step S1, the preparing mobile phase A includes: taking 1 ml of methanoic acid in 1000 ml of water, ultrasonically mixing to obtain the mobile phase A.

In some embodiments of the present disclosure, in step S1, the preparing mobile phase B includes: taking 1 ml methanoic acid in 1000 ml acetonitrile, ultrasonically mixing to obtain the mobile phase B.

In some embodiments of the present disclosure, in step S1, the preparing a 50% acetonitrile solution includes: taking 500 ml of acetonitrile and mixing it in 500 ml of water to obtain the 50% acetonitrile solution.

In some embodiments of the present disclosure, in step S2, the preparing the test solution includes:
weighing 100 mg of natural flavonoid selenium sample, placing it in a 50 ml volumetric flask, adding 50% acetonitrile solution to dilute to a mark, sonicating for 10 minutes, mixing well, filtering to obtain the test solution.

In some embodiments of the present disclosure, in step S3, the testing the test solution includes: taking 10 μl of the test sample solution and injecting it into a dual ternary high performance liquid chromatograph, measuring and recording a chromatogram.

In some embodiments of the present disclosure, in step S3, a model of the dual ternary high-performance liquid chromatography is U3000-CAD.

In summary, due to the above technical solution, the beneficial effects of the present disclosure are as following.

In the present disclosure, a method for detecting natural flavonoid selenium is provided to ensure direct quantification of a quality control in drug development and application. Through a standard curve, a compensation effect of a dual ternary pump can be utilized to ensure that a response value of each substance passing through a detector correspond to an amount of the substance, without being affected by fluidity gradients and without the need to prepare standard samples again.

In the present disclosure, the detection of substances that do not have UV absorption is achieved. A synthesis process of natural flavonoid selenium involves complex reactions, resulting in a large amount of substances that are not absorbed by UV. Conventional liquid-phase methods use UV detectors, which cannot fully monitor the reaction products. CAD detectors can detect any non-volatile and partially semi-volatile substances.

### BRIEF DESCRIPTION OF DRAWINGS

FIG. 1 is a flowchart of the present disclosure.

FIG. 2 is a typical schematic diagram of the present disclosure.

FIG. 3 is a schematic diagram of comparison results between a CAD detector and an ultraviolet detector in the present disclosure.

FIG. 4 is a schematic diagram of a comparison of separation results in the present

### DISCLOSURE

### Description of Embodiments

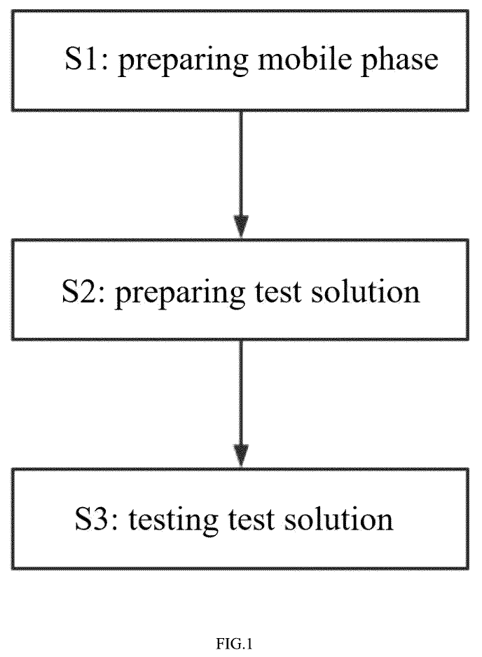

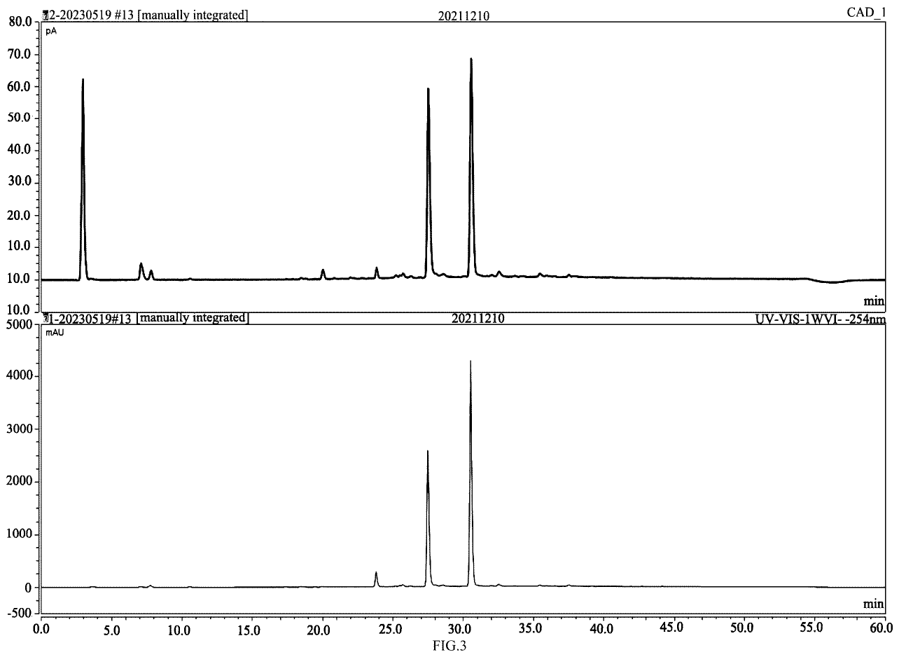

The specific implementation of a method for detecting natural flavonoid selenium of the present disclosure will be further explained in combination with FIGS. 1-4. The method for detecting natural flavonoid selenium in the present disclosure is not limited to the description in the following examples.

### EMBODIMENTS

This embodiment provides a specific implementation mode for detecting natural flavonoid selenium, as shown in FIG. 1, which includes the following steps:
S1: preparing mobile phase;
S2: preparing test solution;
S3: testing the test solution.

In an implementation mode, the mobile phase includes a mobile phase A, a mobile phase B, and a 50% acetonitrile solution.

In an implementation mode, in step S1, the preparing mobile phase A includes: taking 1 ml of methanoic acid in 1000 ml of water, ultrasonically mixing to obtain the mobile phase A.

In an implementation mode, the water is ultrapure water with a specification of 18.2M Ω·cm@25° C.

In an implementation mode, in step S1, the preparing mobile phase B includes: taking 1 ml methanoic acid in 1000 ml acetonitrile, ultrasonically mixing to obtain the mobile phase B.

In an implementation mode, in step S1, the preparing a 50% acetonitrile solution includes: taking 500 ml of acetonitrile and mixing it in 500 ml of water to obtain the 50% acetonitrile solution.

In an implementation mode, in step S2, the preparing the test solution includes: weighing 100 mg of natural flavonoid selenium sample, placing it in a 50 ml volumetric flask, adding 50% acetonitrile solution to dilute to a mark, sonicating for 10 minutes, mixing well, filtering to obtain the test solution.

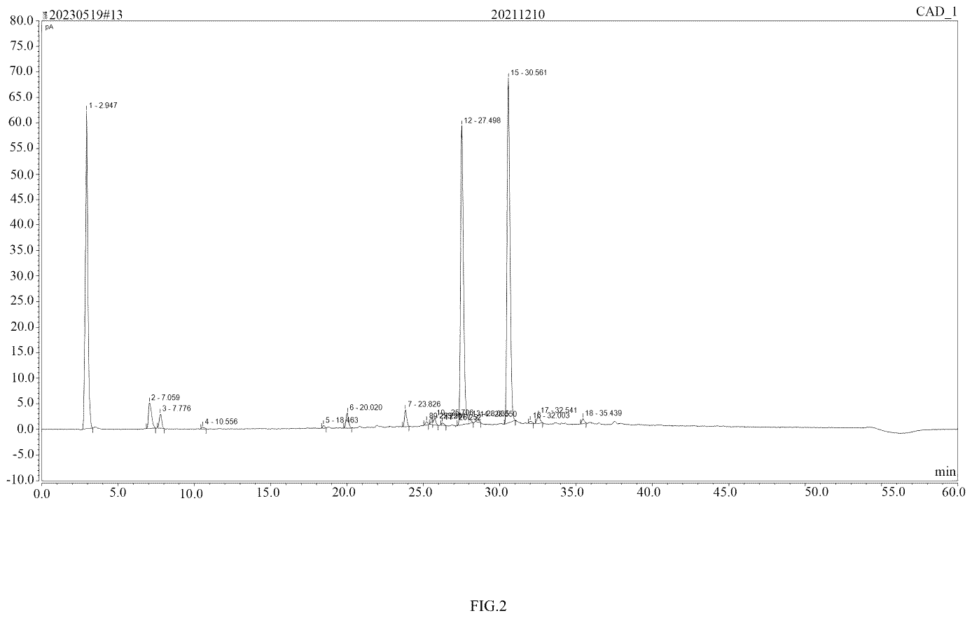

In an implementation mode, in step S3, the testing the test solution includes: taking 10 μl of the test sample solution and injecting it into a dual ternary high performance liquid chromatograph, measuring and recording a chromatogram (as shown in FIG. 2).

In an implementation mode, in step S3, a model of the dual ternary high-performance liquid chromatography is U3000-CAD.

The chromatographic condition is as follows.

| Chromatography Column | ACE Excel 5 C18-PFP 4.6 × 150 mm, 5 μm | | |
|---|---|---|---|
| Mobile phase A | Methanoic acid:water = 1:1000 | | |
| Mobile phase B | Methanoic acid:acetonitrile = 1:1000 | | |
| | Time (min) | % A | % B |
| Analysis pump | 0 | 95 | 5 |
| gradient | 50 | 50 | 50 |
| | 51 | 95 | 5 |
| | 55 | 95 | 5 |
| | Time (min) | % A | % B |
| Compensated pump | 0 | 50 | 50 |
| gradient | 5 | 50 | 50 |
| | 55 | 95 | 5 |
| | 56 | 50 | 50 |
| | 60 | 50 | 50 |

| | |
|---|---|
| 60 | Stop Run |

| | |
|---|---|
| Flow velocity | 0.5 ml/min |
| Column temperature | 25° C. |
| Injection volume | 10 μl |
| Spray temperature of CAD | High (50° C.) |

By adopting the above technical solution.

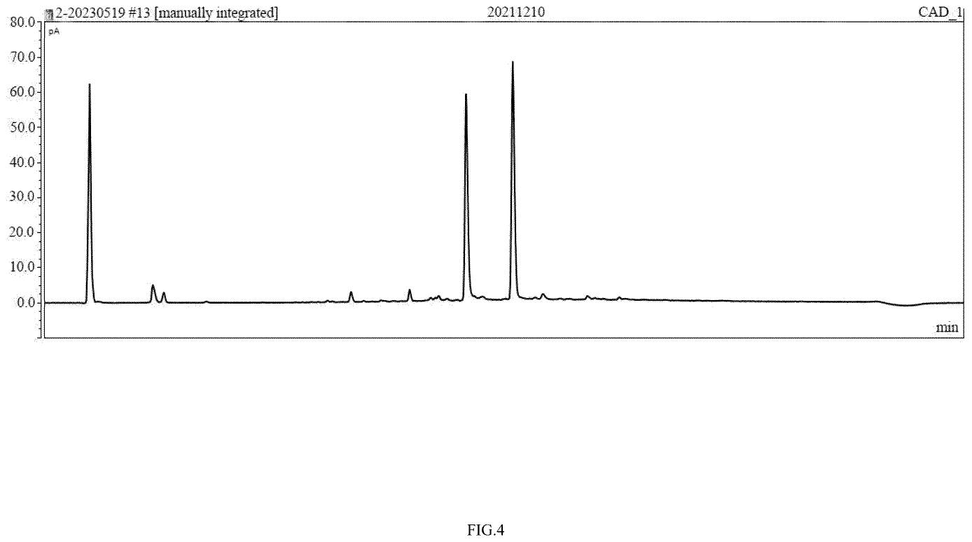

As shown in FIGS. 2-4, it can be seen that in the present application, there are three major breakthroughs in detecting natural flavonoid selenium compared to conventional liquid phase detection.
1. Direct quantification without a need to prepare standard samples. Through a standard curve, this method can utilize a compensation effect of a dual ternary pump, so that a response value of each substance passing through a detector corresponds to an amount of substance, without being affected by the fluidity gradient.
2. The detection of substances without UV absorption was achieved. A synthesis process of natural flavonoid selenium involves complex reactions, resulting in a large amount of substances that are not adsorbed by UV. Conventional liquid-phase methods use UV detectors, which cannot monitor the entire reaction product. The CAD detector in this method is a universal detector that can detect any non-volatile and partially semi-volatile substances.
3. The separation of substances was achieved by using ACE Excel 5 C18-PFP chromatography column and a mobile phase with a certain gradient to effectively separate two substances with high content and similar polarity in natural flavonoid selenium, as shown in FIG. 4 for comparative effect.

The above content is a further detailed explanation of the present disclosure based on preferred embodiments, and it cannot be assumed that the specific implementation of the present disclosure is limited to these explanations. For those skilled in the art to which the present disclosure belongs, several simple deductions or substitutions can be made without departing from the concept of the present disclosure, which should be considered as within the protection scope of the present disclosure.

What is claimed is:

1. A method for detecting natural flavonoid selenium, comprising the following steps:
S1: preparing mobile phase;
S2: preparing test solution;
S3: testing the test solution.

2. The method for detecting natural flavonoid selenium as claimed in claim 1, wherein in step S1, the mobile phase comprises a mobile phase A, a mobile phase B, and a 50% acetonitrile solution.

3. The method for detecting natural flavonoid selenium as claimed in claim 2, wherein in step S1, the preparing mobile phase A comprises: taking 1 ml of methanoic acid in 1000 ml of water, ultrasonically mixing to obtain the mobile phase A.

4. The method for detecting natural flavonoid selenium as claimed in claim 2, wherein in step S1, the preparing mobile phase B comprises: taking 1 ml methanoic acid in 1000 ml acetonitrile, ultrasonically mixing to obtain the mobile phase B.

5. The method for detecting natural flavonoid selenium as claimed in claim 2, wherein in step S1, the preparing a 50% acetonitrile solution comprises: taking 500 ml of acetonitrile and mixing it in 500 ml of water to obtain the 50% acetonitrile solution.

6. The method for detecting natural flavonoid selenium as claimed in claim 1, wherein in step S2, the preparing test solution comprises:
weighing 100 mg of natural flavonoid selenium sample, placing it in a 50 ml volumetric flask, adding 50% acetonitrile solution to dilute to a mark, sonicating for 10 minutes, mixing well, filtering to obtain the test solution.

7. The method for detecting natural flavonoid selenium as claimed in claim 1, wherein in step S3, the testing the test solution comprises: taking 10 μl of the test sample solution and injecting it into a dual ternary high performance liquid chromatograph, measuring and recording a chromatogram.

8. The method for detecting natural flavonoid selenium as claimed in claim 7, wherein in step S3, a model of the dual ternary high-performance liquid chromatography is U3000-CAD.
